(19) United States
(12) Patent Application Publication
(10) Pub. No.: US 2007/0196407 A1
(43) Pub. Date: Aug. 23, 2007

(54) Alkoxyphenylcarboxylic acid derivatives

(76) Inventors: Werner Holzl, Eschentzwiller (FR); Andrea Preuss, Basel (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/589,383

(22) PCT Filed: Feb. 11, 2005
(86) PCT No.: PCT/EP05/50605

§ 371 Date: Aug. 15, 2006

(30) Foreign Application Priority Data
Feb. 20, 2004 (EP) 04100674.3

(51) Int. Cl. A61K 31/265

(52) U.S. Cl. 424/405; 424/400; 424/401; 424/49; 424/65; 424/76.1; 514/512

(57) ABSTRACT

Use of compounds of formula (I) wherein R₁ is substituted or unsubstituted C₈-C₃₀alkyl or substituted or unsubstituted C₃-C₇cyclo-alkyl, and R₂ is H, COOH or substituted or unsubstituted O-C₁-C₃₀alkyl, in the antimicrobial treatment of surfaces, in the preservation of cosmetics, household products, textiles and plastics, and for use in disinfectants.

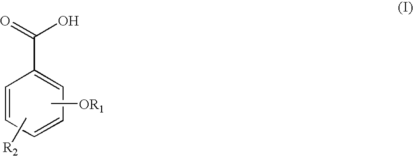

The present invention relates to the use of alkoxybenzenecarboxylic acid derivatives in the antimicrobial treatment of surfaces, in the preservation of cosmetics, household products, textiles and plastics, and for use in disinfectants.

The present invention relates to the use of compounds of formula (I)

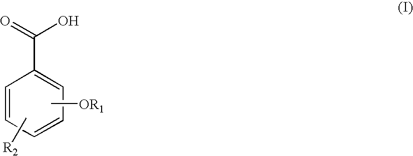

wherein

R₁ is substituted or unsubstituted C₁-C₃₀alkyl or substituted or unsubstituted C₃-C₇cyclo-alkyl, and

R₂ is H, COOH or substituted or unsubstituted O-C₁-C₃₀alkyl,

in the antimicrobial treatment of surfaces.

Preference is given to the use according to the invention of compounds of formula (I) wherein

R₁ is substituted or unsubstituted C₁-C₂₀alkyl or substituted or unsubstituted C₃-C₇cyclo-alkyl, and

R₂ is H, COOH or substituted or unsubstituted O-C₁-C₂₀alkyl.

Special preference is given to the use according to the invention of compounds of formula (I) wherein

R₁ is substituted or unsubstituted C₈-C₂₀alkyl, and

R₂ is H, COOH or substituted or unsubstituted O-C₈-C₂₀alkyl; and very special preference is given to the use according to the invention of compounds of formula (I) wherein

R₁ is substituted or unsubstituted C₈-C₂₀alkyl, and

R₂ is H or COOH.

C₁-C₃₀Alkyl, C₁-C₂₀alkyl, C₈-C₂₀alkyl, O-C₁-C₃₀alkyl, O-C₁-C₂₀alkyl and O-C₈-C₂₀alkyl are straight-chained or branched and are uninterrupted or interrupted one or more times by at least one of the radicals selected from the group of the unsaturated hydrocarbon radicals and hetero atoms: —CR₃=CR₄— and O, S, CO, N, NR₅,

wherein R₃, R₄ and R₅ are each independently hydrogen or C₁-C₄alkyl.

For example, alkyl interrupted by O is:

—CH₂CH₂—O—CH₂CH₂—O—CH₂CH₂—OCH₂CH₂—O—CH₂CH₂—O—CH₂CH₃, or —CH₂CH₂—O—CH₂CH₃, or —CH₂CH₂—O—CH₂CH₂—O—CH₂CH₂—O—CH₂CH₃, or —CH₂CH₂—O—CH₂CH₂—CH₂CH₂—O—CH—(CH₃)₂.

For example, alkyl interrupted by N is:

—CH₂CH₂CH₂CH₂CH₂CH₂CH₂CH₂N(CH₃)₂, —CH₂CH₂CH₂CH₂CH₂CH₂—NH—CH₃, —(CH₂)₇—N(CH₃)—CH₃, —(CH₂)₇—NH—CH₃, —CH₂CH₂CH₂CH₂—NH—CH₂CH₂CH₂CH₃, or —CH₂CH₂CH₂CH₂N(CH₃)—CH₂CH₂CH₂CH₃.

Substituents of C₁-C₃₀alkyl, C₁-C₂₀alkyl, C₈-C₂₀alkyl, O-C₁-C₃₀alkyl, O-C₁-C₂₀alkyl and —O-C₈-C₂₀alkyl are, for example, C₁-C₄alkyl, halide or CN.

C₁-C₃₀Alkyl is, for example, methyl, ethyl, n-propyl, isopropyl, n-butyl, sec-butyl, tert-butyl, n-pentyl, 2-pentyl, 3-pentyl, 2,2′-dimethylpropyl, n-hexyl, n-heptyl, n-octyl, 1,1′,3,3′-tetramethyl-butyl, 2-ethylhexyl, nonyl, decyl, undecyl, dodecyl, tridecyl, tetradecyl, pentadecyl, hexadecyl, heptadecyl, octadecyl, nonadecyl, eicosanyl or tricosanyl.

C₁-C₂₀Alkyl is, for example, methyl, ethyl, n-propyl, isopropyl, n-butyl, sec-butyl, tert-butyl, n-pentyl, 2-pentyl, 3-pentyl, 2,2′-dimethylpropyl, n-hexyl, n-heptyl, n-octyl, 1,1′,3,3′-tetramethyl-butyl, 2-ethylhexyl, nonyl, decyl, undecyl, dodecyl, tridecyl, tetradecyl, pentadecyl, hexadecyl, heptadecyl, octadecyl, nonadecyl or eicosanyl.

C₈-C₂₀Alkyl is, for example, n-octyl, 1,1′,3,3′-tetramethylbutyl, 2-ethylhexyl, nonyl, decyl, undecyl, dodecyl, tridecyl, tetradecyl, pentadecyl, hexadecyl, heptadecyl, octadecyl, nona-decyl or eicosanyl.

C₁-C₄Alkyl is, for example, methyl, ethyl, n-propyl, isopropyl, n-butyl, sec-butyl or tert-butyl.

—O-C₁-C₃₀Alkyl is, for example, methoxy, ethoxy, n-propoxy, isopropoxy, n-butoxy, sec-butoxy, tert-butoxy, n-pentyloxy, 2-pentyloxy, 3-pentyloxy, 2,2′-dimethylpropoxy, n-hexyloxy, n-heptyloxy, n-octyloxy, 1,1′,3,3′-tetramethylbutoxy, 2-ethylhexyloxy, nonyloxy, decyloxy, undecyloxy, dodecyloxy, tridecyloxy, tetradecyloxy, pentadecyloxy, hexadecyloxy, hepta-decyloxy, octadecyloxy, nonadecyloxy, eicosanyloxy or tricosanyloxy.

O-C₁-C₂₀Alkyl is, for example, methoxy, ethoxy, n-propoxy, isopropoxy, n-butoxy, sec-butoxy, tert-butoxy, n-pentyloxy, 2-pentyloxy, 3-pentyloxy, 2,2′-dimethylpropoxy, n-hexyloxy, n-heptyloxy, n-ctyloxy, 1,1′,3,3′-tetramethylbutoxy, 2-ethylhexyloxy, nonyloxy, decyloxy, undecyloxy, dodecyloxy, tridecyloxy, tetradecyloxy, pentadecyloxy, hexadecyloxy, hepta-decyloxy, octadecyloxy, nonadecyloxy or eicosanyloxy.

O-C₈-C₂₀Alkyl is, for example, n-octyloxy, 1,1′,3,3′-tetramethylbutoxy or 2-ethylhexyloxy, nonyloxy, decyloxy, undecyloxy, dodecyloxy, tridecyloxy, tetradecyloxy, pentadecyloxy, hexadecyloxy, heptadecyloxy, octadecyloxy, nonadecyloxy or eicosanyloxy.

C₃-C₇Cycloalkyl is, for example, cyclopropyl, cyclobutyl, cyclopentyl, cyclohexyl or cyclo-heptyl.

Halide is, for example, fluoride, bromide, chloride or iodide and preferably chloride.

In the use according to the invention, examples of very especially preferred compounds correspond to formulae

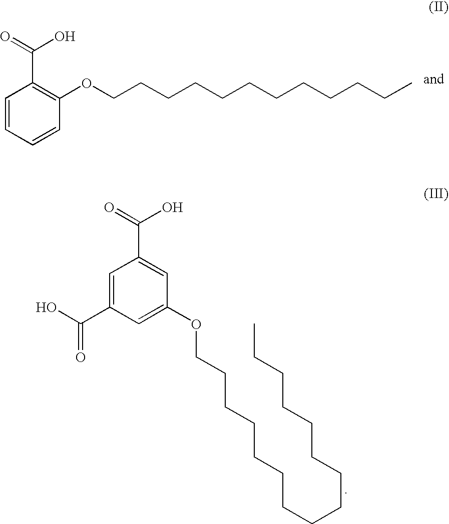

The alkoxybenzenecarboxylic acid derivatives according to the invention are prepared according to processes known per se, generally by alkylating carboxylic acid esters of hydroxy-carboxylic acids, preferably the methyl esters, in suitable solvents, such as ethanol, aceto-nitrile, dimethylformamide or acetone, with the appropriate alkyl halides in the presence of an organic or inorganic base, preferably potassium carbonate, and then hydrolysing the alkoxy-carboxylic acid esters to form the free acids:

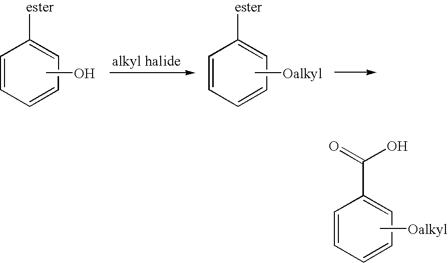

The alkoxybenzenecarboxylic acid derivatives of formula (I) according to the invention exhibit pronounced antimicrobial activity, especially against gram-positive and gram-negative bacteria and also against bacteria of the skin flora, additionally against yeasts and moulds. They are therefore especially suitable for the disinfection, deodorisation and general and antimicrobial treatment of the skin and mucosa and also of integumentary appendages (hair), very especially for the disinfection of the hands and of wounds.

The alkoxybenzenecarboxylic acid derivatives of formula (I) according to the invention are therefore suitable as antimicrobial active ingredients and preservatives in personal care preparations, for example shampoos, bath additives, hair-care preparations, liquid and solid soaps (based on synthetic surfactants and salts of saturated and/or unsaturated fatty acids), lotions and creams, deodorants, other aqueous or alcoholic solutions, e.g. cleansing solutions for the skin, moist cleansing cloths, oils or powders.

The invention relates to a personal care preparation comprising at least one compound of formula (I) as well as cosmetically tolerable carriers or adjuvants.

The present invention relates also to the use of a compound of formula (I) in the antimicrobial treatment, deodorisation and disinfection of the skin, oral and other mucosa, the surfaces of the teeth, and hair.

The present invention relates also to the use of a compound of formula (I) in the disinfection and deodorisation of surfaces.

The personal care preparation according to the invention comprises

a) from 0.01 to 15% by weight, preferably from 0.1 to 10% by weight, based on the total weight of the preparation, of the compound of formula (I) and

b) cosmetically tolerable adjuvants.

Depending on the form of the personal care preparation it also comprises, in addition to the alkoxybenzenecarboxylic acid derivative of formula (I), further constituents, for example sequestering agents, colourants, perfume oils, thickeners or setting agents (consistency regulators), emollients, UV absorbers, skin-protecting agents, antioxidants, additives that improve the mechanical properties, such as dicarboxylic acids and/or aluminium, zinc, calcium and magnesium salts of C₁₄-C₂₂ fatty acids, and, optionally, preservatives.

The personal care preparation according to the invention may be formulated as a water-in-oil or oil-in-water emulsion, as an alcoholic or alcohol-containing formulation, as a vesicular dispersion of an ionic or non-ionic amphiphilic lipid, as a gel, as a solid stick or as an aerosol formulation.

In the case of a water-in-oil or oil-in-water emulsion, the cosmetically tolerable adjuvant contains preferably from 5 to 50% of an oily phase, from 5 to 20% of an emulsifier and from 30 to 90% water. The oily phase may contain any oil suitable for cosmetic formulations, e.g. one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Cosmetic formulations according to the invention are used in a variety of fields. Especially the following preparations, for example, come into consideration:
skin-care preparations, e.g. skin-washing and cleansing preparations in the form of tablet-form or liquid soaps, soapless detergents or washing pastes;
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose and pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, nail-care preparations, such as nail varnish, nail varnish removers, nail hardeners or cuticle removers;
intimate hygiene preparations, e.g. intimate washing lotions or intimate sprays;
foot-care preparations, e.g. foot baths, foot powders, foot creams or foot balsams, special deodorants and antiperspirants or callus-removing preparations;
light-protective preparations, such as sun milks, lotions, creams and oils, sun blocks or tropicals, pre-tanning preparations or after-sun preparations;
skin-tanning preparations, e.g. self-tanning creams;
depigmenting preparations, e.g. preparations for bleaching the skin or skin-lightening preparations;
insect repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. soapless detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or after-shave lotions;
fragrance preparations, e.g. fragrances (eau de Cologne, eau de toilette, eau de parfum, parfum de toilette, perfume), perfume oils or cream perfumes;
dental-care, denture-care and mouth-care preparations, e.g. toothpastes, gel toothpastes, tooth powders, mouthwash concentrates, anti-plaque mouthwashes, denture cleaners or denture fixatives;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, foams, hairsprays, bleaching preparations, e.g. hydrogen peroxide solutions, lightening shampoos, bleaching creams, bleaching powders, bleaching pastes or oils, temporary, semi-permanent or permanent hair colourants, preparations containing self-oxidising dyes, or natural hair colourants, such as henna or camomile.

An antimicrobial soap has, for example, the following composition:

0.01 to 5% by weight of the compound of formula (I)

0.3 to 1% by weight titanium dioxide

1 to 10% by weight stearic acid and

ad 100% soap base, e.g. the sodium salts of tallow fatty acid and coconut fatty acid or glycerol.

A shampoo has, for example, the following composition:

0.01 to 5% by weight of the compound of formula (I)

12.0% by weight sodium laureth-2-sulfate

4.0% by weight cocamidopropyl betaine

3.0% by weight NaCl and

water ad 100%.

A deodorant has, for example, the following composition:

0.01 to 5% by weight of the compound of formula (I)

60% by weight ethanol

0.3% by weight perfume oil and

water ad 100%.

The invention relates also to an oral composition comprising

0.01 to 15% by weight, based on the total weight of the composition, of the compound of formula (I), and orally tolerable adjuvants.

Example of an oral composition:

10% by weight sorbitol

10% by weight glycerol

15% by weight ethanol

15% by weight propylene glycol

0.5% by weight sodium lauryl sulfate

0.25% by weight sodium methylcocyl taurate

0.25% by weight polyoxypropylene/polyoxyethylene block copolymer

0.10% by weight peppermint flavouring

0.1 to 0.5% by weight of a compound of formula (I) and water ad 100%.

The oral composition according to the invention may be, for example, in the form of a gel, a paste, a cream or an aqueous preparation (mouthwash).

The oral composition according to the invention may also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name Olafluor.

The present invention relates also to the use of the compounds of formula (I) in the preservation of surfaces.

The present invention relates also to the use of the compounds of formula (I) in the antimicrobial treatment, especially the preservation, of textile fibre materials. Such materials are natural and dyed or printed fibre materials, for example silk, wool, polyamide or poly-urethanes, and especially all kinds of cellulosic fibre materials. Such fibre materials include, for example, natural cellulose fibres, such as cotton, linen, jute and hemp, and also cellulose and regenerated cellulose. Preferred suitable textile fibre materials are those of cotton.

The present invention relates also to the use of the compounds of formula (I) in the antimicrobial treatment of plastics, especially in imparting antimicrobial properties to or preserving plastics, plastics being, for example, polyethylene, polypropylene, polyurethane, polyester, polyamide, polycarbonate and latex. Fields of application therefor are, for example, floor coverings, plastics coatings, plastics containers and packaging materials; kitchen and bathroom utensils (e.g. brushes, shower curtains, sponges, bath mats), latex, filter materials (air and water filters), plastics articles used in the medical field, e.g. dressings, syringes, catheters etc., so-called medical devices, gloves and mattresses.

The present invention relates also to the use of the compounds of formula (I) in the antimicrobial treatment of paper, e.g. toilet paper, nonwovens, e.g. nappies, sanitary towels, panty liners and cloths for the hygiene and household sector.

The present invention relates also to the use of the compounds of formula (I) for antimicrobial treatment in washing and cleaning formulations, e.g. in liquid and powder detergents or softeners.

The present invention relates also to the use of the compounds of formula (I) for antimicrobial treatment in household and general purpose cleaners for the cleaning and disinfection of hard surfaces.

A cleaning agent has, for example, the following composition:

| | |
|---|---|
| 0.01 to 5% | of the compound of formula (I) |
| 3.0% | octyl alcohol 4EO |
| 1.3% | fatty alcohol C₈-C₁₀ polyglucoside |
| 3.0% | isopropanol |
| ad 100% | water. |

In addition to the preservation of cosmetics and household products, it is also possible to use the compounds of formula (I) in preserving and imparting antimicrobial properties to commercial products, such as printing thickeners of starch or cellulose derivatives, surface coatings and paints; and to use them as a biocide in industrial processes, for example in the treatment of paper, especially in paper-treatment liquors.

The present invention relates also to the use of the compounds of formula (I) in the antimicrobial treatment of wood and in the antimicrobial treatment of leather, in the preservation of leather and in imparting antimicrobial properties to leather.

The present invention relates also to the use of the compounds of formula (I) in protecting cosmetic products and household products against microbial damage.

The present invention relates also to the non-therapeutic use of the compounds of formula (I) in inhibiting or destroying microorganisms that form a biofilm. Compounds of formula (I) are suitable for penetrating biofilms on living and inanimate surfaces; for preventing the adhesion of bacteria to surfaces and optionally preventing the further build-up of the biofilm; for removing the biofilm and/or inhibiting the further growth of or destroying the microorganisms in the biological matrix that form the biofilm.

The term “biofilm” refers very generally to aggregates of living and dead microorganisms, especially bacteria, adhering to living and inanimate surfaces, in conjunction with their metabolic products in the form of extracellular polymeric substances (EPS matrix), e.g. polysaccharides. The activity of antimicrobial active ingredients, which normally exhibit pronounced growth-inhibiting or destroying action in respect of planktonic cells, can be greatly reduced in respect of microorganisms that are organised into biofilms, for example because of inadequate penetration of the active ingredient into the biomatrix.

In the case of the present invention, this relates especially to biofilms on the surfaces of the teeth and on the oral mucosa of humans, which biofilms, as a result of the microorganisms forming them or their metabolic products, play a decisive part in the development of degenerative diseases in the oral cavity, for example caries or periodontitis.

The Examples which follow illustrate the present invention but do not limit it.

### EXAMPLE 1

### Preparation of 2-dodecyloxy-benzoic acid (II)

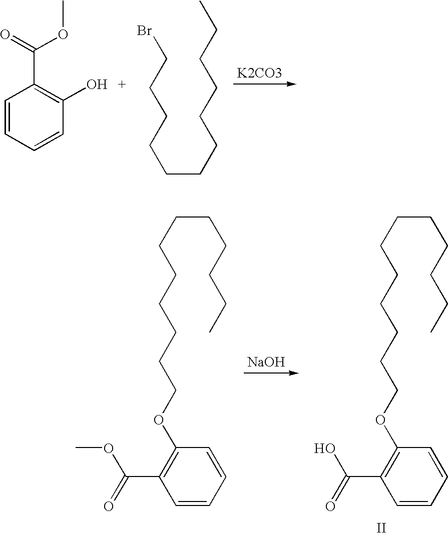

40.2 ml (312 mmol) of 2-hydroxybenzoic acid methyl ester are placed in acetonitrile with 47.4 g (343 mmol) of potassium carbonate, under a nitrogen atmosphere, and heated for one hour under reflux (white suspension). Then, after the addition of 89.8 ml (374 mmol) of dodecyl bromide and 5.2 g (31 mmol) of potassium iodide, stirring is carried out for 48 hours under reflux.

For working up, the solvent is drawn off and the colourless oily-solid residue is suspended in water and extracted by shaking with diethyl ether. The ether phase is washed with 1N NaOH and water and dried over Na₂SO₄. After drawing off the solvent, an oily-solid residue remains, which is recrystallised from methanol.

Colourless crystals, yield: 48.1 g (48% of theory).

For hydrolysis of the ester, 48.1 g (150 mmol) of the methyl ester are suspended in a solution of 25.2 g (450 mmol) of KOH in 265 ml of water, and the suspension is heated for 10 hours under reflux (colourless solution). After dilution with 300 ml of water, the mixture is acidified to pH 1 with 6N HCl and the colourless precipitate is isolated, washed and dried.

Colourless crystals, yield: 42.0 g (91% of theory).

### EXAMPLE 2

### Preparation of 5-hexadecyloxy-isophthalic acid (III)

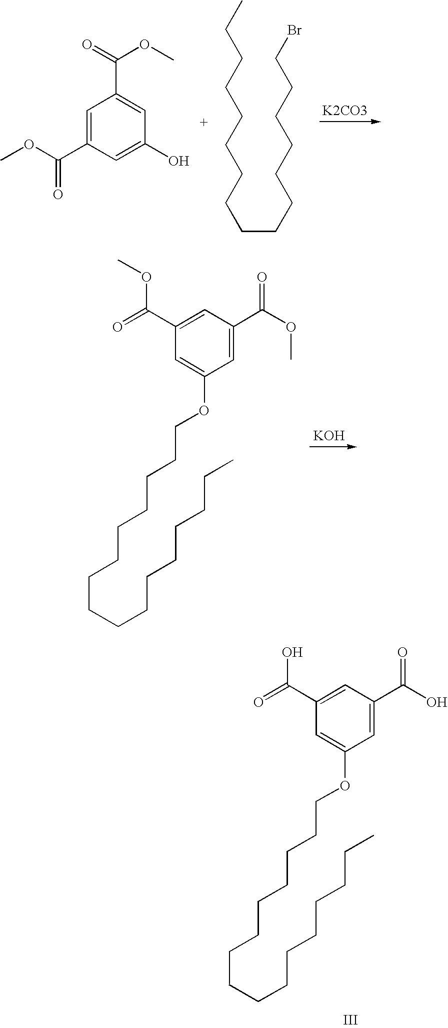

24.0 g (114 mmol) of 5-hydroxy-isophthalic acid dimethyl ester are suspended together with 17.3 g (125 mmol) of potassium carbonate in 375 ml of acetonitrile and heated for one hour at 80° C. under a nitrogen atmosphere. 34.8 ml (114 mmol) of hexadecyl bromide are then added, and stirring is carried out for 24 hours at 80° C. under a nitrogen atmosphere. For working up, the reaction mixture is concentrated and filtered; 250 ml of water are added to the white residue, and extraction is carried out by shaking with 175 ml of diethyl ether 3 times. After conventional working up of the organic phase, the product remains behind in sufficient purity for further reaction.

Colourless solid, yield 44.4 g (90% of theory).

For hydrolysis of the ester, 90.5 g of 5-hexadecyloxy-isophthalic acid dimethyl ester are suspended in a solution of 45.9 g of KOH in 500 ml of water, and the suspension is heated for 14 hours under reflux. 6N HCl is then added to the mixture, while it is still hot, until the reaction is strongly acidic, and the mixture is cooled and then filtered. The residue is washed thoroughly with water several times and dried.

Colourless solid, yield 62.6 g (73% of theory).

### EXAMPLE 3

### Determination of the Minimum Inhibitory Concentration (MIC) in the Agar Incorporation Test (MIC Test)

Medium: casein-soybean flour peptone agar (Merck)
*Sabouraud 4% glucose agar (Merck)

Dilution medium: sterile 0.85% NaCl solution

Test organisms: Staphylococcus aureus ATCC 6853 and 9144

Escherichia coli ATCC 10536 and NCTC 8196

Pseudomonas aemuginosa ATCC 15422
*Candida albicans ATCC 10231
*Aspergillus niger ATCC 6275

Incubation: 24 hours at 37° C.
*3 days at 28° C.

Test solution: 1% stock solutions of all the test substances are prepared in a suitable solvent and diluted in serial dilutions as far as possible so that final concentrations of from 500 ppm to 10 ppm are obtained in the agar.

Test principle:

0.3 ml of the particular dilution stage is mixed with 15 ml of still liquid nutrient medium. After solidification of the nutrient base, 10 μl of the test strains in a suitable organism dilution in 0.85% NaCl solution are applied point-wise to the agar medium:

| | |
|---|---|
| Staphylococcus aureus ATCC 6538 | 1:100 dilution |
| Escherichia coli ATCC 10536 | 1:1000 dilution |
| Pseudomonas aeruginosa ATCC 15442 | 1:1000 dilution |
| Candida albicans ATCC 10231 | 1:10 dilution |
| Aspergillus niger ATCC 6275 | 1:10 dilution |

TABLE 1

| | 2-Dodecyloxy- | 5-Hexadecyloxy- |
|---|---|---|
| | benzoic acid | isophthalic acid |
| Microorganism | (II) | (III) |
| Staphylococcus aureus ATCC 6538 | 31.25 | 24 |
| Staphylococcus aureus ATCC 9144 | 31.25 | 24 |
| Staphylococcus epidermidis | 15.63 | 12 |
| ATCC 12228 | | |
| Corynebacterium minutissimum | 15.63 | 24 |
| ATCC 23348 | | |
| Propionibacterium acnes | 15.63 | 12 |
| ATCC 6919 (*) | | |
| Escherichia coli NCTC 8196 | >500 | >48 |
| Escherichia coli ATCC 10536 | >500 | >48 |
| Proteus vulgaris ATCC 6896 | >500 | >48 |
| Klebsiella pneumoniae ATCC 4352 | >500 | >48 |
| Salmonella choleraesuis | >500 | >48 |
| ATCC 9184 | | |
| Pseudomonas aeruginosa | >500 | >48 |
| ATCC 15442 | | |
| Candida albicans ATCC 10231 | >500 | >48 |
| Aspergillus niger ATCC 6275 | >500 | >48 |

### EXAMPLE 4

### Determination of the MIC Against Various Oral Organisms in the Serial Dilution Test

Medium: Thioglycolate broth with hemin and menadione
Columbia broth with hemin and menadione for P. gingivalis and P. nigrescens

Dilution medium: The appropriate amount of the substances was pipetted directly onto the medium.

Test organisms: Actinobacillus actinomycetemcomitans ATCC 43718
Streptococcus gordonii ATCC 10558
Streptococcus mutans ATCC 33402
Actinomyces viscosus ATCC 43146
Fusobacterium nucleatum subsp. polymorphum ATCC 10953
Porphyromonas gingivalis ATCC 33277
Prevotella nigrescens ATCC 33563

Incubation: 7-10 days at 37° C. anaerobic, or 24 h aerobic with 10% CO₂for Streptococci and A. actinomycetemcomitans

Test solution: Stock solutions of all the test substances in ethanol comprising 1500 ppm (w/w) are used.

Test principle:

Bacteria are removed from blood agar plates using cotton-wool-tipped sticks and a suitable O.D. is established in the appropriate medium (McFarland 0.5); this solution is used undiluted for F. nucleatum and P. nigrescens and diluted 1:20 for the other strains. 0.1 ml of bacteria culture is added to 2 ml of each of the active substance solutions, and incubation is carried out as described above.

TABLE 2

| | 2- | 5- |
|---|---|---|
| | Dodecyloxy- | Hexadecyloxy- |
| | benzoic | isophthalic |
| Microorganism | acid (II) | acid (III) |
| A. actinomycetemcomitans ATCC43718 | >15 | 15 |
| S. gordonii ATCC10558 | 15 | ≦0.94 |
| S. mutans ATCC33402 | 7.5 | 3.75 |
| A. viscosus ATCC43146 | 1.88 | 1.88 |
| F. nucleatum subsp. polymorphum | >15 | >15 |
| ATCC10953 | | |
| P. gingivalis ATCC3277 | 7.5 | ≦0.94 |
| P. nigrescens ATCC33563 | >15 | >15 |

### EXAMPLE 5

### Test of the Substantivity of 2-dodecyloxy-benzoic Acid (II) and 5-hexadecyloxy-isophthalic Acid (III) on Hydroxyapatite and Determination of the Growth-Inhibiting Action

Principle:

apatite plates (dia. 10 mm) are placed in synthetic saliva (Deutsche Zahnärztliche Zeitschrift DZZ May 2002), rinsed in NaCl and then incubated in an ethanolic solution of test substances (II) and (III) comprising 50 and 500 ppm. The plates are then introduced into a Nunclon Surface titre plate (12 wells) and incubated for 24 hours at 37° C. in Caso nutrient medium which has been inoculated with Actinomyces viscosus ATCC 43146 (about 10⁵/ml).

Result:

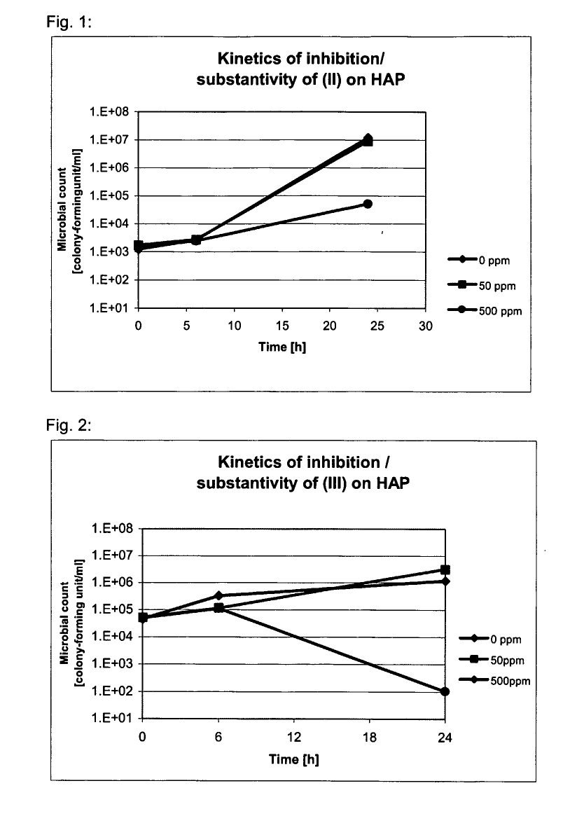

FIGS. 1 and 2 show the inhibition of the growth of Actinomyces viscosus by 2-dodecyloxy-benzoic acid (II) and 5-hexadecyloxy-isophthalic acid (III) after adsorption of the substance onto hydroxyapatite plates pretreated with synthetic saliva, in comparison with an untreated control.

### EXAMPLE 6

### Test of the Activity of 2-dodecyloxy-benzoic Acid (II) in the Biofilm Model

Principle: see Guggenheim et a/. (2001), “Validation of an in vitro biofilm model of supragingival plaque”, J. Dent Res. 80 (1)

Hydroxyapatite discs are pretreated with human saliva and a biofilm of a defined mixed culture of various gram+ and gram− oral organisms (Streptococci, Actinomyces sp., Veillonella & Fusobacterium sp. & others) and a yeast (Candida albicans) are allowed to grow on the hydroxyapatite. During the test period of 4 days, the discs are immersed, several times in all, in the below given application formulation of the substance (II),

| | |
|---|---|
| sodium laurylsulfate | 1.00% |
| ethanol | 20.00% |
| substance (II) | 0.5% |
| PEG-40 hydrogenated Castor Oil | 5.00% |
| popylene Glycol | 10.00% |
| water | 63.5% |

and at the end the biofilm is harvested and the number of living cells is determined.

Result:

The results of two independent tests are shown in Table 3. The alkoxybenzene-carboxylic acid brings about a marked reduction in the microbial count of the biofilm on the hydroxy-apatite discs compared with the placebo formulation without active substance.

TABLE 3

| | 2-Dodecyloxy- | |
|---|---|---|
| | benzoic acid (II) | |
| Sample | (in mouthwash | Control |
| (mean values) | formulation) | (placebo formulation) |
| [CFU/disc] | 1.2 × 10⁵ | 5.1 × 10⁷ |
| CFU = colony-forming unit | 1.7 × 10⁵ | 7.9 × 10⁷ |

1. A method for the antimicrobial treatment of substrates by applying onto or incorporating into said substrate a composition comprising a compound of formula (I) 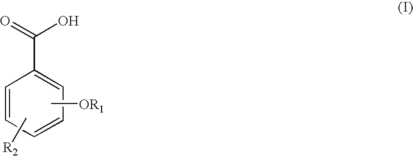
wherein
R₁ is substituted or unsubstituted C₆-C₃₀alkyl or substituted or unsubstituted C₃-C₇cycloalkyl, and
R₂ is H, COOH or substituted or unsubstituted O-C₁-C₃₀alkyl.

2. A method according to claim 1, wherein R₁ is substituted or unsubstituted C₈-C₂₀alkyl.

3. A method according to claim 1, wherein a composition comprising a compound of formula (I) is used applied to skin, oral or other mucosa, surfaces of teeth, or hair.

4. A method according to claim 1, wherein a composition comprising a compound of formula (I) provides for disinfection or deodorisation of a substrate.

5. A method according to claim 1 wherein a composition comprising a compound of formula (I) is applied to textile fibre materials.

6. (canceled)

7. A method according to claim 1, wherein a compound of formula (I) is incorporated into washing and cleaning formulations.

8. A method according to claim 1 for imparting antimicrobial properties to plastics, paper, nonwovens, wood or leather.

9. A method according to claim 1 for preserving commercial products.

10. A method according to claim 1, wherein a compound of formula (I) is incorporated as a biocide in an industrial process.

11. A personal care preparation comprising
a) from 0.01 to 15% by weight, based on the total weight of the preparation, of a compound of formula (I) defined according to claim 1, and
b) cosmetically tolerable adjuvants.

12. An oral composition comprising
a) from 0.01 to 15% by weight, based on the total weight of the composition, of a compound of formula (I) defined according to claim 1, and
b) orally tolerable adjuvants.

13. A method according to claim 1 for preventing the build-up of a biofilm of microorganisms and/or in removing a biofilm and/or in inhibiting or destroying microorganisms that form a biofilm.

14. A method according to claim 1 for the antimicrobial treatment of a surface by applying onto said surface a composition comprising a compound of formula (I).

15. A method according to claim 14 for the disinfection or deodorisation of a surface.

16. A method according to claim 3 for the disinfection or deodorisation of skin, oral or other mucosa, surfaces of the teeth, or hair.

17. A method according to claim 9 for preserving plastics, paper, nonwovens, wood or leather.
